(19) United States
(12) Patent Application Publication
(10) Pub. No.: US 2024/0100222 A1
(43) Pub. Date: Mar. 28, 2024

(54) COATED SUBSTRATE AND METHOD FOR FORMING COATED SUBSTRATE

(71) Applicant: THE SECANT GROUP, LLC, Telford, PA (US)

(72) Inventors: Mohamed A. ELKHODIRY, Lansdale, PA (US); Andrew METZGER, Lafayette Hill, PA (US); Amanda K. WEBER, Macungie, PA (US); Stephanie REED, Conshohocken, PA (US)

(21) Appl. No.: 18/470,599

(22) Filed: Sep. 20, 2023

Related U.S. Application Data
(60) Provisional application No. 63/408,989, filed on Sep. 22, 2022.

(51) Int. Cl. A61L 27/34; A61L 27/54; A61L 27/56; D03D 15/283; D03D 15/50; D04H 1/4358; D04H 1/56; D04H 1/728; D06N 3/00; D06N 3/12

(52) U.S. Cl. CPC A61L 27/34; A61L 27/54; A61L 27/56; D03D 15/283; D03D 15/50; D04H 1/4358; D04H 1/56; D04H 1/728; D06N 3/0006; D06N 3/0011; D06N 3/0056; D06N 3/009; D06N 3/123; A61L 2430/20; D06N 2201/0281; D06N 2209/12; D10B 2331/04; D10B 2331/10; D10B 2401/063; D10B 2401/10; D10B 2509/00

(57) ABSTRACT

A coated substrate is disclosed, including a solvent-sensitive substrate and a polyester coating disposed on the solvent-sensitive substrate, wherein the polyester coating includes a polyester copolymer of a polyol and a polyacid. A method for forming a coated substrate is disclosed including spraying solvated polyester material from a spray nozzle onto a solvent-sensitive substrate and drying the solvated polyester material to form a polyester disposed on the solvent-sensitive substrate, wherein the solvated polyester material and the polyester coating include a polyester copolymer of a polyol and a polyacid.

### RELATED APPLICATIONS

This application claims the benefit of and priority to U.S. Provisional Patent Application No. 63/408,989, filed Sep. 22, 2022, entitled “Anisotropic Textiles, Devices, and Implants and Methods for Forming and Coated Articles and Methods for Forming Coated Articles,” which is hereby incorporated by reference in its entirety.

### FIELD OF THE INVENTION

This application is directed to coated substrates and methods for forming coated substrates. In particular, this application is directed to coated substrates with a polyester or polyester urethane coating disposed on a solvent-sensitive substrate and methods for forming coated substrates with a polyester coating disposed on the solvent-sensitive substrate.

### BACKGROUND OF THE INVENTION

Current prosthetic heart valves carry a significant level of risk to patients and are often associated with durability issues. Mechanical heart valves are prone to thrombosis, and their rigid structure disrupts natural hemodynamics. Bioprosthetic heart valves result in native-like hemodynamics but are hampered with calcification and leaflet degeneration issues that limit the valve durability and require repeated surgery. In addition, bioprosthetic valves rely on chemically treating bovine or porcine animal tissue, a process that carries a considerable regulatory burden, high cost of manufacturing, and scalability issues. The chemical treatment of bovine or porcine animal tissue can consist of a variety of processes, such as decellularization, removal of antigenic components from the xenograft, and fixation of the natural biopolymer tissue to extend shelf life and stability. Fixation typically involves crosslinking of collagen or gelatin natural biopolymers using formaldehyde or glutaraldehyde. This crosslinking of collagen or gelatin can render the materials stiffer, more immunogenic, and more difficult to degrade.

Synthetic heart valves fabricated from chemically defined polymers may replace native valve functions with tunability, cost-effectiveness, and scalability. However, unlike native heart valve leaflets, thin polymer films are isotropic. Thin polymer films also often have low tear strength and suboptimal fatigue profile. These films need to match strict mechanical requirements that may often be at odds with function in hemodynamic conditions. Soft elastic materials often lack sufficient tear strength and durability while tough, durable materials are often too stiff for valve functions.

In comparison, textiles distribute loads over a network and maintain excellent flexure properties while having a higher tear strength; however, current textiles are also typically isotropic and lack a mechanical profile that resembles native heart valve leaflets and their functions in terms of directional elastic modulus, flexure, and creep.

Existing technologies for providing a directional mechanical profile focus on fiber alignment of electro-spun fibers, a technology that is difficult to scale and control compared to textile manufacturing. In addition, textiles are already integrated into the manufacturing of current prosthetic heart valves.

Weaving with elastic thermoplastic polyurethane is challenging; known methods lack control of the elongation of the thermoplastic polyurethane while weaving.

Additionally, thermoplastic polyurethanes are incompatible with many organic solvent-based secondary processes, as thermoplastic polyurethanes are soluble in a number of solvents. While solubility of thermoplastic polyurethanes in various solvents may improve processing of thermoplastic polyurethanes, this solubility simultaneously limits the ability of thermoplastic polyurethanes to be exposed to the same solvents in downstream manufacturing. As such, applying solvent-based coatings in downstream secondary processes to textiles formed with thermoplastic polyurethanes may be an obstacle, and it is often desirable to avoid applying any solvents to thermoplastic polyurethane fibers that may augment, solubilize, degrade, or reduce any properties or features of the thermoplastic polyurethane fibers.

It would be desirable in the art to have textile-based materials not having the aforementioned drawbacks.

### BRIEF DESCRIPTION OF THE INVENTION

In one exemplary embodiment, a coated substrate includes a solvent-sensitive substrate and a polyester coating disposed on the solvent-sensitive substrate, wherein the polyester coating includes a polyester copolymer of a polyol and a polyacid.

In another exemplary embodiment, a method for forming a coated substrate includes spraying solvated polyester material from a spray nozzle onto a solvent-sensitive substrate and drying the solvated polyester material to form a polyester coating disposed on the solvent-sensitive substrate, wherein the solvated polyester material and the polyester coating include a polyester copolymer of a polyol and a polyacid.

Further aspects of the subject matter of the present disclosure are provided by the following clauses:

A coated substrate including a solvent-sensitive substrate and a polyester coating disposed on the solvent-sensitive substrate, wherein the polyester coating includes a polyester copolymer of a polyol and a polyacid.

The coated substrate of any preceding clause, wherein the solvent-sensitive substrate is selected from the group consisting of polyurethanes, thermoplastic polyurethanes, polyacrylates, rubbers, low density polyethylenes, high density polyethylenes, polycarbonates, polypropylenes, polystyrenes, acrylonitrile butadiene styrenes, poly(L-lactic acid)s, poly(D,L-lactic acid)s, polycaprolactones, poly(delta-valerolactone)s, poly(ethylene glycol)s, poly(glycerol sebacate)s, poly(glycerol sebacate) urethanes, poly(glycerol sebacate)-alginates, poly(glycerol sebacate) urethane-alginates, polydimethylsiloxanes, polycaprolactone-polyurethane blends, polycaprolactones, poly(lactic-co-glycolic acid)s, poly(glycolic acid)s, styrene block copolymers, polyvinylidene fluorides, and combinations thereof.

The coated substrate of any preceding clause, wherein the substrate is selected from the group consisting of a fiber, a yarn, a textile, an anisotropic textile, a plurality of electrospun fibers, an electrospun mat, a film, a membrane, a three-dimensional scaffold, a device or device component, and combinations thereof.

The coated substrate of any preceding clause, wherein the solvent-sensitive substrate is a yarn.

The coated substrate of any preceding clause, wherein the solvent-sensitive substrate is a textile.

The coated substrate of any preceding clause, wherein the solvent-sensitive substrate is an anisotropic textile.

The coated substrate of any preceding clause, wherein the anisotropic textile includes warp yarn having an elastic modulus of 0.2 MPa to 50 MPa and weft yarn having an elastic modulus of 50 MPa to 500 MPa, and wherein the elastic modulus of the warp yarn is at least 50% higher than the elastic modulus of the weft yarn.

The coated substrate of any preceding clause, wherein polyester coating reduces porosity of the textile.

The coated substrate of any preceding clause, wherein the polyester coating is a conformal coating maintaining porosity of the textile.

The coated substrate of any preceding clause, wherein the solvent-sensitive substrate is a plurality of electrospun fibers.

The coated substrate of any preceding clause, wherein the solvent-sensitive substrate is an electrospun mat.

The coated substrate of any preceding clause, wherein the polyester coating reduces porosity of the electrospun mat.

The coated substrate of any preceding clause, wherein the polyester coating is a conformal coating maintaining porosity of the electrospun mat.

The coated substrate of any preceding clause, wherein the substrate is a film.

The coated substrate of any preceding clause, wherein the substrate is a three-dimensional scaffold.

The coated substrate of any preceding clause, wherein the substrate is a device or device component.

The coated substrate of any preceding clause, wherein the device or the device component is selected from the group consisting of a heart-valve leaflet, a heart valve replacement implant, a transcatheter valve replacement system, a cardiac patch, a vascular seal, a ligament repair, a tendon repair, an organ tissue engineering scaffold, a hernia mesh, an adhesion barrier, a dura mater repair membrane, a dura mater replacement membrane, a muscle repair, a rotor cuff repair, a sensor, a wearable sensor, a wound dressing, a dermal patch, a cosmetic patch, a wearable patch, a drug delivery device, a wearable drug delivery device, and combinations thereof.

The coated substrate of any preceding clause, wherein the polyester coating further includes at least one therapeutic agent.

The coated substrate of any preceding clause wherein the polyester coating further includes an additive selected from the group consisting of animal-derived proteins, human-derived proteins, recombinant proteins, synthetic extracellular matrix proteins, peptides, proteoglycans, glycosaminoglycans, polysaccharides, surfactants, thixotropic agents, plasticizers, nanoparticles, leveling agents, wetting agents, electrical conduction agents, stabilizers, lubricants, antioxidants, and combinations thereof.

The coated substrate of any preceding clause, further including an electro-spun mesh disposed on the solvent-sensitive substrate.

The coated substrate of any preceding clause, wherein a boundary between the solvent-sensitive substrate and the polyester coating is an integrated interphase boundary in which the polyester coating infiltrates or intermixes with the solvent-sensitive substrate.

The coated substrate of any preceding clause, wherein a boundary between the solvent-sensitive substrate and the polyester coating is a non-integrated interface boundary in which the polyester coating is essentially free of intermixing with or infiltration into the solvent-sensitive substrate.

The coated substrate of any preceding clause, wherein the polyester copolymer is selected from the group consisting of poly(glycerol sebacate)s, poly(glycerol sebacate) urethanes, poly(glycerol adipate)s, poly(glycerol adipate) urethanes, poly(glycerol suberate)s, poly(glycerol suberate) urethanes, poly(glycerol succinate)s, poly(glycerol succinate) urethanes, poly(pentane-diol sebacate)s, poly(pentane-diol sebacate) urethanes, poly(pentane-diol adipate)s, poly(pentane-diol adipate) urethanes, poly(pentane-diol suberate)s, poly(pentane-diol suberate) urethanes, poly(pentane-diol succinate)s, poly(pentane-diol succinate) urethanes, copolymers thereof, thermally-cured polymers thereof, and combinations thereof.

The coated substrate of any preceding clause, wherein the polyester copolymer includes a polyester urethane formed with a diisocyanate selected from the group consisting of hexamethylene diisocyanate, lysine diisocyanate, and combinations thereof.

A method for forming a coated substrate including spraying solvated polyester material from a spray nozzle onto a solvent-sensitive substrate and drying the solvated polyester material to form a polyester coating disposed on the solvent-sensitive substrate, wherein the solvated polyester material and the polyester coating include a polyester copolymer of a polyol and a polyacid.

The method of any preceding clause, wherein a distance between the spray nozzle and the solvent-sensitive substrate is at least 12 cm.

The method of any preceding clause, wherein the distance between the spray nozzle and the solvent-sensitive substrate is at least 15 cm.

The method of any preceding clause, wherein the solvated polyester material is sprayed at a flow rate less than 1 mL/min.

The method of any preceding clause, wherein the flow rate is 0.5 mL/min or less.

The method of any preceding clause, wherein the solvated polyester material is sprayed for 10 continuous passes or less.

The method of any preceding clause, wherein the solvated polyester material is solvated in 100% acetone.

### BRIEF DESCRIPTION OF THE DRAWINGS

FIG. 1A depicts the surface topology of an anisotropic textile before poly(glycerol sebacate) urethane coating, according to an embodiment of the present disclosure.

FIG. 1B depicts the surface topology of an anisotropic textile after poly(glycerol sebacate) urethane coating, according to an embodiment of the present disclosure.

FIG. 2A depicts the surface topology of an electro-spun mat before poly(glycerol sebacate) urethane coating, according to an embodiment of the present disclosure.

FIG. 2B depicts the surface topology of an electro-spun mat after two cycles of poly(glycerol sebacate) urethane coating, according to an embodiment of the present disclosure.

FIG. 2C depicts the surface topology of an electro-spun mat after four cycles of poly(glycerol sebacate) urethane coating, according to an embodiment of the present disclosure.

FIG. 2D depicts the surface topology of an electro-spun mat after ten cycles of poly(glycerol sebacate) urethane coating, according to an embodiment of the present disclosure.

FIG. 3 depicts anisotropic heart valve replacement implant leaflets, according to an embodiment of the present disclosure.

FIG. 4 depicts a mechanical profile of an anisotropic textile, according to an embodiment of the present disclosure.

Wherever possible, the same reference numbers will be used throughout the drawings to represent the same parts.

### DETAILED DESCRIPTION OF THE INVENTION

Embodiments of the present disclosure, in comparison to coated substrates and methods for forming coated substrates lacking one or more of the features of the present invention, may decrease costs of production, decrease potential points of failure in the resulting devices, provide practical customizability, extend service life, or combinations thereof.

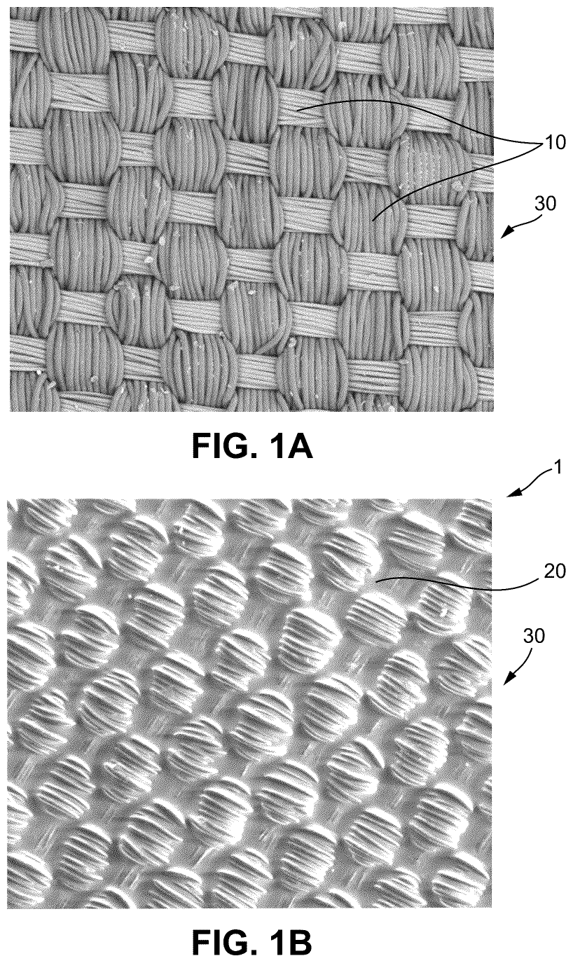

Referring to FIGS. 1A-B and 2A-D, in one embodiment a coated substrate 1 includes a solvent-sensitive substrate 10 and a polyester coating 20 disposed on the solvent-sensitive substrate 10.

As used herein, a “solvent sensitive substrate” refers to a substrate formed of a material which dissolves upon sustained contact for less than 12 hours with FDA class 3 solvents, such as, but not limited to, acetone, propyl acetate, ethyl acetate, methyl acetate, butanol, butyl acetate, pentanol, and propanol, such that the solvent sensitive substrate loses more than 15% of any of its mass, overall dimension, or surface topography.

The polyester coating 20 disposed on the solvent-sensitive substrate 10 comprises a polyester copolymer of a polyol and a polyacid. As used herein, a “a polyester copolymer of a polyol and a polyacid” refers to polyester polymers formed by copolymerization of at least one polyol and at least one diacid. The polyester may be crosslinked by a diisocyanate. The polyester copolymer of a polyol and a polyacid may be a bioresorbable polymer. The polyol may be any suitable species, including, but not limited to, glycerol, pentane diol, butane diol, propane diol, isopentyl diol, ethylene glycol, sorbitol, xylitol, or combinations thereof. The polyacid may be any suitable species, including, but not limited to, a diacid. Suitable diacids include, but are not limited to, sebacic acid, diacids having a shorter chain-length than sebacic acid, such as, but not limited to, adipic acid, succinic acid, suberic acid, or combinations thereof, or combinations of any of the foregoing diacids.

The solvent-sensitive substrate 10 may be formed of any suitable material, including, but not limited to, polyurethanes, thermoplastic polyurethanes, polyacrylates, rubbers, low density polyethylenes, high density polyethylenes, polycarbonates, polypropylenes, polystyrenes, acrylonitrile butadiene styrenes, poly(L-lactic acid)s, poly(D,L-lactic acid)s, polycaprolactones, poly(delta-valerolactone)s, poly(ethylene glycol)s, poly(glycerol sebacate)s, poly(glycerol sebacate) urethanes, poly(glycerol sebacate)-alginates, poly(glycerol sebacate) urethane-alginates, polydimethylsiloxanes, polycaprolactone-polyurethane blends, polycaprolactones, poly(lactic-co-glycolic acid)s, poly(glycolic acid)s, styrene block copolymers, polyvinylidene fluorides, or combinations thereof.

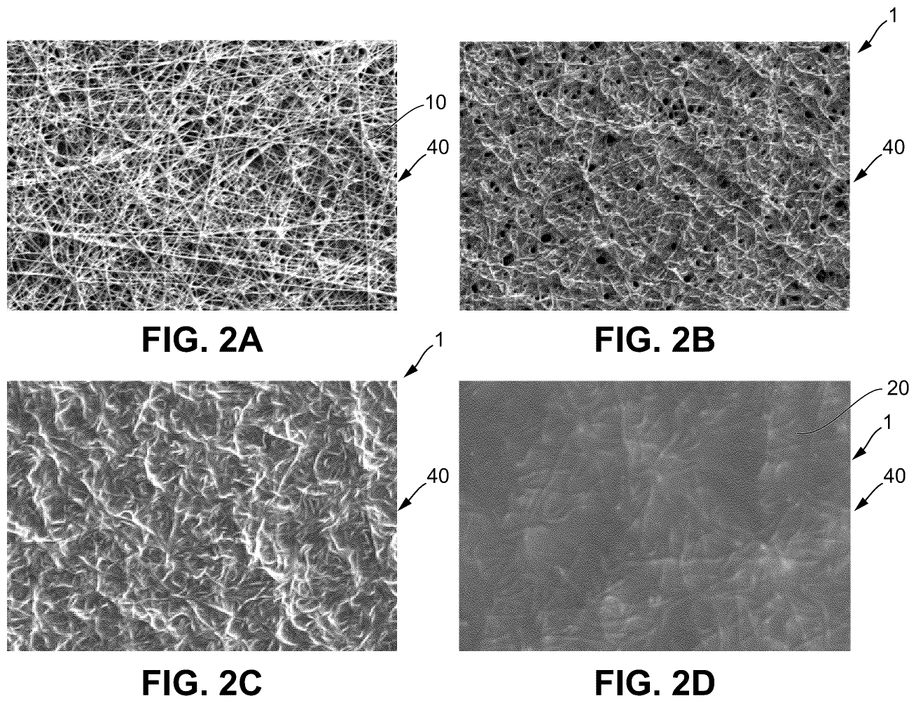

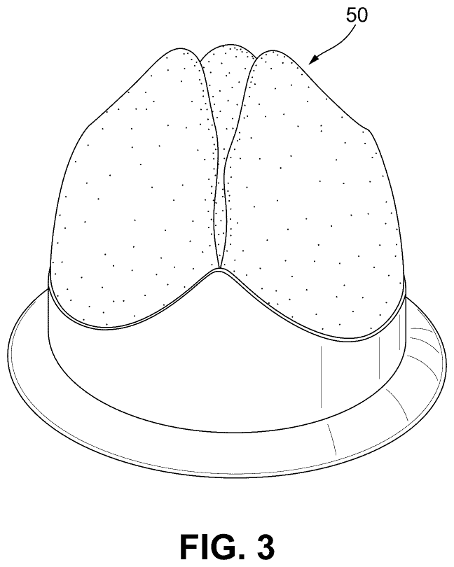

The solvent-sensitive substrate 10 may be any suitable article, including, but not limited to, a fiber, a yarn, a textile 30 (FIGS. 1A-B), an anisotropic textile, a plurality of electrospun fibers, an electrospun mat 40 (FIGS. 2A-D), a film, a membrane, a three-dimensional scaffold, a device or device component, or combinations thereof. Suitable devices or device components include, but are not limited to, heart-valve leaflets 50 (FIG. 3), heart valve replacement implants, transcatheter valve replacement systems, cardiac patches, vascular seals, ligament repairs, tendon repairs, organ tissue engineering scaffolds, hernia meshes, adhesions barriers, dura mater repair membranes, dura mater replacement membranes, muscle repairs, rotor cuff repairs, sensors, wearable sensors, wound dressings, dermal patches, cosmetic patches, wearable patches, drug delivery devices, wearable drug delivery devices, or combinations thereof. The heart-valve leaflets 50 may include a single leaflet, two leaflets, three leaflets, or any suitable larger number of leaflets.

As used herein, “textile” is construed broadly. The generally accepted and common definition of textile derived articles of manufacture is any material or component made of interlacing filament, fiber, extrudate or multifilament yarn. A textile component product or goods may be defined in any of the following ways: raw, semi-worked, worked, semi-manufactured, manufactured, semi-made up, or made-up products composed of textile components. A “textile,” as referenced herein, may be a component or finished good. “Textile” and “textile component,” are not used herein limited to the traditional resource or connotation implying fiber-based “fabric” or cloth. “Textile” and “textile component” may include any use of resources to a designed-end that involves fiber, threads, film-fibers, extruded sheets, composites, yarns, or filament-based raw material components further engineered or processed into a two-dimensional or three-dimensional interim or final formed article using known textile form-factor engineering techniques, including, but not limited to, knitting, braiding, weaving, non-woven processing, staple, film, laminate sheeting, extrusion, film building, or composite multilayer processing. This includes medical-surgical exogenous and endogenous implant devices such as tissue engineered scaffold-graft forms and planar articles such as leaflets 50. These planar articles may be further processed, but are not limited to, die-punch, cutting, stamping, or laser processing. Textiles and textile components may be made from diverse materials which have been derived from distinctive primary sources such as, but not limited to, natural animals, processed animals, natural plants/vegetables, processed plants/vegetables, inorganic minerals (such as, but not limited to, metals and oxides), or inorganic polymers (such as, but not limited to, carbon fibers, glasses, and bio glasses), regenerated man-made manufactured or synthetic/laboratory derived resources and manufactured or bio-derived recombinant synthetic and biopolymer resources. Furthermore, “textile engineering” is considered to be an area of material form-factor engineering that uses such natural and or synthetic materials processing, scientific and material engineering principles, and polymer chemical principles to produce or improve the designed article, components or products, or materials including, but not limited to, medical devices as well as other commercially recognized “textile” components. The textiles described or referenced herein may be considered permanent synthetic, environmentally non-degradable, non-biodegradable, biodegradable, or bioresorbable. Biodegradable and bioresorbable may include, but are not limited to, lactides, glycolides, glycerol esters, or other degradable bio polymers, including, but not limited to, polymers decomposed under aerobic or anaerobic conditions by action of microorganisms, humoral enzymatic attack, aided hydrolysis, or combinations thereof.

By using two different fibers, one soft and one strong, in two perpendicular directions of the fabric, the resulting textile may have biomimetic anisotropy. As used herein, “soft” indicates an elastic modulus of less than 50 MPa, “strong” indicates an elastic modulus greater than 50 MPa, and an elastic modulus of 50 MPa is regarded as a transition point between “soft” and “strong.” This mechanical anisotropy may promote efficient bending, opening, and closing while maintaining high tear strength. This textile anisotropy may mimic the fiber orientation and mechanical performance of collagen and elastin in native tissue leaflets 50. During systole, blood flow pushes leaflets 50 open and causes corrugations on the back side of the leaflet. When leaflets 50 are open during systole, elastin fibers are relaxed while collagen fibers are crimped, compressed, and unaligned. During diastole, back pressure pushes leaflets 50 closed. When leaflets 50 are closed during diastole, elastin fibers are elongated and stretched while collagen fibers are aligned and uncrimped. Elastic fibers may be approximated by soft elastomeric synthetic fibers and collagen fibers may be approximated by strong synthetic fibers.

Using a textile that combines two different fibers with different mechanical properties as the backbone of the valve leaflet 50 may mimic the anisotropy observed in native valve tissue. Anisotropy of mechanical properties facilitates valve functioning and long-term durability. Using a soft yarn in the axial direction may facilitate valve leaflet 50 opening and closing, while using a tough yarn in the circumferential direction may maintain the overall mechanical integrity, longevity, and tear strength of the leaflets 50. Combining the fabric with a polyester coating may also enhance the mechanical properties of synthetic valve leaflets 50 by creating a tough-soft composite instead of plain polymer film. The mechanical flexibility of the fiber used in the warp (axial direction) may promote the ability of the structure to open and close in response to changes in blood pressure while being fixed to the valve base/delivery system. A sequence of warp yarns may be employed, each with varying flexibility to enhance regions of pliancy in the axial direction. This technique may also be employed in the weft (circumferential) direction where varying fiber tenacities may be beneficial, therefore building in regions of tunable strength.

In one embodiment, the coated substrate 1 is an anisotropic textile 30 including warp yarn having an elastic modulus of 0.2 MPa to 50 MPa and weft yarn having an elastic modulus of 50 MPa to 500 MPa, wherein the elastic modulus of the warp yarn is at least 50% higher than the elastic modulus of the weft yarn. The warp yarn or the weft yarn or both may have a non-circular cross-section. For embodiments with yarn having an elastic modulus of 0.2 MPa to 50 MPa, the elastic modulus may be any suitable sub-range thereof, including, but not limited to, 0.2 MPa to 0.5 MPa, alternatively 0.2 MPa to 1.0 MPa, alternatively 0.5 MPa to 1.5 MPa, alternatively 1 MPa to 2 MPa, alternatively 1.5 MPa to 2.5 MPa, alternatively 0.2 MPa to 10 MPa, alternatively 2 MPa to 15 MPa, alternatively 5 MPa to 15 MPa, alternatively 10 MPa to 20 MPa, alternatively 15 MPa to 25 MPa, alternatively 20 MPa to 30 MPa, alternatively 25 MPa to 35 MPa, alternatively 30 MPa to 40 MPa, alternatively 35 MPa to 45 MPa, alternatively 40 MPa to 50 MPa, subranges thereof, or combinations thereof. Alternatively, a hyperelastic yarn may be substituted that would have an equivalent elastic modulus under physiological strain (between 10-30% strain). For embodiments with yarn having an elastic modulus of 50 MPa to 500 MPa, the elastic modulus may be any suitable sub-range thereof, including, but not limited to, 50 MPa to 150 MPa, alternatively 50 MPa to 100 MPa, alternatively 75 MPa to 125 MPa, alternatively 90 MPa to 150 MPa, alternatively 100 MPa to 150 MPa, alternatively 100 MPa to 200 MPa, alternatively 150 MPa to 250 MPa, alternatively 200 MPa to 300 MPa, alternatively 250 MPa to 350 MPa, alternatively 300 MPa to 400 MPa, alternatively 350 MPa to 450 MPa, alternatively 400 MPa to 500 MPa, subranges thereof, or combinations thereof.

The weft yarn may be formed of any suitable weft material, including, but not limited to, a weft material selected from the group consisting of polyethylene terephthalate, polyether ether ketone, polyether ketone ketone [just checking, should this be stated twice?], polyvinylidene fluoride, polypropylene, poly(glycolic acid), poly(lactic-co-glycolic acid), ultra-high-molecular-weight polyethylene, and combinations thereof. The warp yarn may be formed of any suitable warp material, including, but not limited to, a warp material selected from the group consisting of thermoplastic poly-urethane, polyurethane, poly(glycerol sebacate), poly(glycerol sebacate) urethane, poly(glycerol sebacate)-alginate, poly(glycerol sebacate) urethane-alginate. polydimethylsiloxane, polycaprolactone-polyurethane blend, styrene block copolymer, and combinations thereof.

In a substrate which is porous, such as, but not limited to, a textile 30, a membrane, or an electrospun mat 40, the polyester coating 20 may reduce the porosity of the solvent-sensitive substrate 10 or may be a conformal coating maintaining the porosity of the solvent-sensitive substrate 10.

In one embodiment, the polyester coating 20 further includes at least one therapeutic agent.

The polyester coating 20 may further include additives, including, but not limited to, animal-derived proteins, human-derived proteins, recombinant proteins, synthetic extracellular matrix proteins, peptides, proteoglycans, glycosaminoglycans, polysaccharides, surfactants, thixotropic agents, plasticizers, nanoparticles, leveling agents, wetting agents, electrical conduction agents, stabilizers, lubricants, antioxidants, or combinations thereof.

In one embodiment, the coated substrate 1 further includes an electro-spun mesh disposed on the solvent-sensitive substrate 10.

The boundary between the solvent-sensitive substrate 10 and the polyester coating 20 may be an integrated interphase boundary in which the polyester coating 20 infiltrates or intermixes with the solvent-sensitive substrate 10 or may be a non-integrated interface boundary in which the polyester coating 20 is essentially free of intermixing with or infiltration into the solvent-sensitive substrate 10. As used herein, “essentially free” indicates that less than 10% of the surface area at the boundary is free of intermixing or infiltration. Without being bound by theory, it is believed that an integrated interphase boundary increases bonding forces between the solvent-sensitive substrate 10 and the polyester coating 20, reducing the likelihood of interfacial failure modes such as delamination and cracking.

The polyester copolymer may have any suitable material composition, including, but not limited to, poly(glycerol sebacate)s, poly(glycerol sebacate) urethanes, poly(glycerol adipate)s, poly(glycerol adipate) urethanes, poly(glycerol suberate)s, poly(glycerol suberate) urethanes, poly(glycerol succinate)s, poly(glycerol succinate) urethanes, poly(pentane-diol sebacate)s, poly(pentane-diol sebacate) urethanes, poly(pentane-diol adipate)s, poly(pentane-diol adipate) urethanes, poly(pentane-diol suberate)s, poly(pentane-diol suberate) urethanes, poly(pentane-diol succinate)s, poly(pentane-diol succinate) urethanes, copolymers thereof, thermally-cured polymers thereof, or combinations thereof. In one embodiment, the polyester copolymer includes a polyester urethane formed with a diisocyanate selected from the group consisting of hexamethylene diisocyanate, lysine diisocyanate, and combinations thereof.

In one embodiment, a method for forming a coated substrate 1, includes spraying solvated polyester material from a spray nozzle onto a solvent-sensitive substrate 10 and drying the solvated polyester material to form a polyester coating 20 disposed on the solvent-sensitive substrate 10.

The spray nozzle may be disposed at any suitable distance from the solvent-sensitive substrate 10 during application of the solvated polyester material, including a distance of at least 12 cm, alternatively at least 15 cm, alternatively at least 18 cm, alternatively between 12 cm to 24 cm.

The solvated polyester material may be sprayed onto the solvent-sensitive substrate 10 at any suitable flow rate, including, but not limited to, a flow rate of less than 1 mL/min, alternatively less than 0.75 mL/min, alternatively less than 0.5 mL/min, alternatively less than 0.25 mL/min, alternatively less than 0.10 mL/min.

The solvated polyester material may be sprayed onto the solvent-sensitive substrate 10 for any suitable number of continuous passes, including, but not limited to, 10 continuous passes or less, alternatively 9 continuous passes or less, alternatively 8 continuous passes or less, alternatively 7 continuous passes or less, alternatively 6 continuous passes or less, alternatively 5 continuous passes or less.

The solvated polyester material may be solvated in any suitable solvent, including, but not limited to, 100% acetone, an FDA class 3 solvent having equal or higher volatility than acetone, or combinations thereof, rather than a 1:1 mixture of acetone and propyl acetate.

Without being bound by theory, it is believed that a polyester coating 20 applied by spray coating outperforms application by dip coating with respect to improved adhesion. It is further believed that improved control of the amount of solvent that touches the solvent-sensitive substrate 10 in a spray process relative to a dip process leads to the further improved adhesion. Specifically, during spray coating, the aerosolized solvated polyester droplets travel through the air with a predetermined time of flight before landing on the polyester material or solvent-sensitive substrate 10. This promotes some of the solvent to evaporate before reaching the solvent-sensitive substrate 10, and, overall, less solvent is delivered to the solvent-sensitive substrate 10 than with dip coating. With dip coating, there is more solvent present and contacting the solvent-sensitive substrate 10 for longer periods of time, thus increasing the impact of organic solvent solubilizing and negatively affecting the solvent-sensitive substrate 10.

If a solvent-sensitive substrate 10 is merely dip-coated in a polyester material solution or spray-coated using a standard spray technique, the solvent-sensitive substrate 10 may dissolve and lose its native geometry. Optimization of the spray-coating process may include, but is not limited to, increasing the distance between the spraying nozzle and the solvent-sensitive substrate 10 to promote solvent evaporation during transit and reduce solvent accumulation on the solvent-sensitive substrate 10, reducing the flow rate to the spray nozzle, reducing the number of spray-coating passes to promote solvent evaporation, air drying the solvent-sensitive substrate 10 between spray-coating passes, alternating solvents to increase evaporation speed, or combinations thereof.

### EXAMPLES

### Example 1

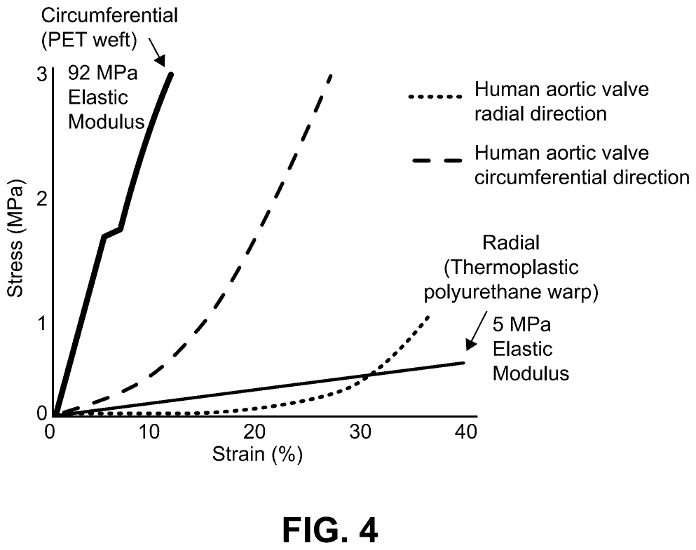

Referring to FIGS. 1A, 1B, and 4, a woven textile consisting of a polyethylene terephthalate weft and a thermoplastic polyurethane (Pellethane 5863-85A from Lubrizol) warp was spray-coated with poly(glycerol sebacate) urethane to form an anisotropic composite structure. A dual-feed Sonotek ultrasonic spray nozzle was loaded with a 15% poly(glycerol sebacate) urethane solution (with 100% acetone as solvent). The solution was sprayed at 0.5 mL/min at a distance of 15 cm from the substrate for 4 cycles. After 24 hours of curing at room temperature, the tensile properties of the composite structure in the weft and warp directions were tested using an MTS CRITERION® 42 mechanical testing system with type v dog-bone samples strained at 10 mm/min. The composite structure had an elastic modulus of 5 MPa in the warp direction and a 92 MPa elastic modulus in the weft direction which indicates its anisotropic properties remained after coating (results shown in FIG. 4 in comparison to a human aortic valve). The elastic moduli were calculated from the slope of the linear portion of the curves measured in FIG. 4. The poly(glycerol sebacate) urethane coated textile was examined under scanning electron microscope, as shown in FIG. 1B, demonstrating the intact underlying architecture of the textile with both thermoplastic polyurethane and thermoplastic polyurethane fibers.

### Example 2

Referring to FIGS. 2A-D, an electrospun mat comprising of thermoplastic polyurethane fibers (Pellethane 5863-85A from Lubrizol) with 1-2 μm fiber diameters and 15-30 μm thickness (FIG. 2A) was spray-coated with poly(glycerol sebacate) urethane to form a composite structure. A dual-feed Sonotek ultrasonic spray nozzle was loaded with a 15% poly(glycerol sebacate) urethane solution (with 100% acetone as solvent). The solution was sprayed at 0.5 mL/min at a distance of 15 cm from the substrate. One electrospun mat was sprayed twice (2 cycles) (FIG. 2B), another was sprayed four times (FIG. 2C), and a third was sprayed ten times (FIG. 2D). As may be observed in FIGS. 2B-D, all three substrates maintained the original architecture of the electrospun mat underneath the poly(glycerol sebacate) urethane layer after 24 hours of curing at room temperature.

While the foregoing specification illustrates and describes exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A coated substrate, comprising:
a solvent-sensitive substrate; and
a polyester coating disposed on the solvent-sensitive substrate,
wherein the polyester coating includes a polyester copolymer of a polyol and a polyacid.

2. The coated substrate of claim 1, wherein the solvent-sensitive substrate is selected from the group consisting of polyurethanes, thermoplastic polyurethanes, polyacrylates, rubbers, low density polyethylenes, high density polyethylenes, polycarbonates, polypropylenes, polystyrenes, acrylonitrile butadiene styrenes, poly(L-lactic acid)s, poly(D,L-lactic acid)s, polycaprolactones, poly(delta-valerolactone)s, poly(ethylene glycol)s, poly(glycerol sebacate)s, poly(glycerol sebacate) urethanes, poly(glycerol sebacate)-alginates, poly(glycerol sebacate) urethane-alginates, polydimethylsiloxanes, polycaprolactone-polyurethane blends, polycaprolactones, poly(lactic-co-glycolic acid)s, poly(glycolic acid)s, styrene block copolymers, polyvinylidene fluorides, and combinations thereof.

3. The coated substrate of claim 1, wherein the solvent-sensitive substrate is selected from the group consisting of a fiber, a yarn, a textile, an anisotropic textile, a plurality of electrospun fibers, an electrospun mat, a film, a membrane, a three-dimensional scaffold, a device or device component, and combinations thereof.

4. The coated substrate of claim 3, wherein the solvent-sensitive substrate includes the anisotropic textile including:
warp yarn having an elastic modulus of 0.2 MPa to 50 MPa; and
weft yarn having an elastic modulus of 50 MPa to 500 MPa, and
wherein the elastic modulus of the warp yarn is at least 50% higher than the elastic modulus of the weft yarn.

5. The coated substrate of claim 3, wherein the solvent-sensitive substrate includes the textile and the polyester coating reduces porosity of the textile.

6. The coated substrate of claim 3, wherein the solvent-sensitive substrate includes the textile and the polyester coating is a conformal coating maintaining porosity of the textile.

7. The coated substrate of claim 3, wherein the solvent-sensitive substrate includes the electrospun mat and the polyester coating reduces porosity of the electrospun mat.

8. The coated substrate of claim 3, wherein the solvent-sensitive substrate includes the electrospun mat and the polyester coating is a conformal coating maintaining porosity of the electrospun mat.

9. The coated substrate of claim 3, wherein the solvent-sensitive substrate includes the device or the device component and the device or the device component is selected from the group consisting of a heart-valve leaflet, a heart valve replacement implant, a transcatheter valve replacement system, a cardiac patch, a vascular seal, a ligament repair, a tendon repair, an organ tissue engineering scaffold, a hernia mesh, an adhesion barrier, a dura mater repair membrane, a dura mater replacement membrane, a muscle repair, a rotor cuff repair, a sensor, a wearable sensor, a wound dressing, a dermal patch, a cosmetic patch, a wearable patch, a drug delivery device, a wearable drug delivery device, and combinations thereof.

10. The coated substrate of claim 1, wherein the polyester coating further includes at least one therapeutic agent.

11. The coated substrate of claim 1, wherein the polyester coating further includes an additive selected from the group consisting of animal-derived proteins, human-derived proteins, recombinant proteins, synthetic extracellular matrix proteins, peptides, proteoglycans, glycosaminoglycans, polysaccharides, surfactants, thixotropic agents, plasticizers, nanoparticles, leveling agents, wetting agents, electrical conduction agents, stabilizers, lubricants, antioxidants, and combinations thereof.

12. The coated substrate of claim 1, further including an electro-spun mesh disposed on the solvent-sensitive substrate.

13. The coated substrate of claim 1, wherein a boundary between the solvent-sensitive substrate and the polyester coating is an integrated interphase boundary in which the polyester coating infiltrates or intermixes with the solvent-sensitive substrate.

14. The coated substrate of claim 1, wherein a boundary between the solvent-sensitive substrate and the polyester coating is a non-integrated interface boundary in which the polyester coating is essentially free of intermixing with or infiltration into the solvent-sensitive substrate.

15. The coated substrate of claim 1, wherein the polyester copolymer is selected from the group consisting of poly(glycerol sebacate)s, poly(glycerol sebacate) urethanes, poly(glycerol adipate)s, poly(glycerol adipate) urethanes, poly(glycerol suberate)s, poly(glycerol suberate) urethanes, poly(glycerol succinate)s, poly(glycerol succinate) urethanes, poly(pentane-diol sebacate)s, poly(pentane-diol sebacate) urethanes, poly(pentane-diol adipate)s, poly(pentane-diol adipate) urethanes, poly(pentane-diol suberate)s, poly(pentane-diol suberate) urethanes, poly(pentane-diol succinate)s, poly(pentane-diol succinate) urethanes, copolymers thereof, thermally-cured polymers thereof, and combinations thereof.

16. The coated substrate of claim 15, wherein the polyester copolymer includes a polyester urethane formed with a diisocyanate selected from the group consisting of hexamethylene diisocyanate, lysine diisocyanate, and combinations thereof.

17. A method for forming a coated substrate, comprising:
spraying solvated polyester material from a spray nozzle onto a solvent-sensitive substrate; and
drying the solvated polyester material to form a polyester coating disposed on the solvent-sensitive substrate
wherein the solvated polyester material and the polyester coating include a polyester copolymer of a polyol and a polyacid.

18. The method of claim 17, wherein a distance between the spray nozzle and the solvent-sensitive substrate is at least 12 cm.

19. The method of claim 17, wherein the solvated polyester material is sprayed at a flow rate less than 1 mL/min.

20. The method of claim 17, wherein the solvated polyester material is sprayed for 10 continuous passes or less.

21. The method of claim 17, wherein the solvated polyester material is solvated in 100% acetone.
